(19) United States
(12) Patent Application Publication
(10) Pub. No.: US 2024/0046440 A1
(43) Pub. Date: Feb. 8, 2024

(54) IMAGE DATA QUALITY EVALUATION METHOD AND APPARATUS, TERMINAL DEVICE, AND READABLE STORAGE MEDIUM

(71) Applicant: SHENZHEN INSTITUTES OF ADVANCED TECHNOLOGY CHINESE ACADEMY OF SCIENCES, Guangdong (CN)

(72) Inventors: Shanshan WANG, Guangdong (CN); Hairong ZHENG, Guangdong (CN); Kehan QI, Guangdong (CN); Chuyu RONG, Guangdong (CN); Xin LIU, Guangdong (CN)

(21) Appl. No.: 18/546,425

(22) PCT Filed: Apr. 14, 2021
(86) PCT No.: PCT/CN2021/087231

§ 371 Date: Aug. 14, 2023

(51) Int. Cl. G06T 7/00; G06T 15/00; G06V 10/44

(52) U.S. Cl. CPC G06T 7/0002; G06T 15/00; G06V 10/44; G06T 2207/20084; G06T 2207/30168

(57) ABSTRACT

An image data quality evaluation method and apparatus, a terminal device, and a readable storage medium. The image data quality evaluation method includes: inputting three-dimensional image data to be evaluated into a feature extraction network model for processing to obtain a feature matrix; processing the feature matrix on the basis of a plurality, of branch network models in a hypemetwork model to obtain a plurality of parameter matrixes, and respectively adjusting, on the basis of the plurality of parameter matrixes, parameters of a plurality of fully connected layers in a corresponding regression model; and processing the feature matrix on the basis of the adjusted regression model to obtain a quality evaluation score. The dynamic adjustment of parameters of a model is implemented on the basis of content of input data, so that the processing efficiency and the quality evaluation precision for three-dimensional image data are improved.

### TECHNICAL FIELD

This application relates to the field of image processing technologies, and specifically to an image data quality evaluation method and apparatus, a terminal device, and a readable storage medium.

### BACKGROUND

With the development of multimedia technology, image data has gradually become the most important way of data dissemination. Correspondingly, the quality of image data also affects all aspects of people's production and life. For example, in the field of medical imaging applications and clinical diagnosis, the quality of medical image data plays a very important role in the quality of medical diagnosis.

Related no-reference image data quality evaluation methods usually include image data quality evaluation methods for specific image distortion types and image data quality evaluation methods for general distortion types. The former has low practical application efficiency, and therefore the latter has become the main research direction of image data quality evaluation-based subjects in recent years.

Commonly used no-reference image data quality evaluation methods based on general distortion types usually suffer from low processing efficiency, low accuracy of quality evaluation results, and the like.

### SUMMARY

### Technical Problem

One of the objectives of the embodiments of this application is to an image data quality evaluation method and apparatus, a terminal device, and a computer-readable storage medium, to resolve the related problem that no-reference image data quality evaluation methods based on general distortion types usually suffer from low processing efficiency and low accuracy of quality evaluation results.

### Solution to the Problem

### Technical Solution

The technical solutions used in the embodiments of this application to resolve the foregoing technical problem are as follows:

According to a first aspect, an image data quality evaluation method is provided, including:

acquiring three-dimensional image data to be evaluated; and

inputting the three-dimensional image data to be evaluated into a pretrained image data quality evaluation network model for processing to obtain a quality evaluation score of the three-dimensional image data to be evaluated, where the pretrained image data quality, evaluation network model includes a feature extraction network model; a hypernetwork model, and a regression model, the feature extraction network model is connected to the hypemetwork model and the regression model, and the hypernetwork model is connected to the regression model, where,

the feature extraction network model is configured to: process the three-dimensional image data to be evaluated to obtain a feature matrix, and send the feature matrix to the hypemetwork model and the regression model; the regression model includes a global maximum pooling layer and a plurality of fully connected layers; the hypemetwork model includes a plurality of branch network models that correspond one to one to the plurality of fully connected layers in the regression model; the hypemetwork model is configured to: process the feature matrix by using the plurality of branch network models to obtain parameter matrixes outputted by the plurality of branch network models, and correspondingly adjust parameters of the plurality of fully connected layers in the regression model according to the parameter matrixes to obtain an adjusted regression model; and the adjusted regression model is configured to process the feature matrix to obtain the quality evaluation score of the three-dimensional image data to be evaluated,

In an embodiment, the branch network model is formed by successively connecting two convolutional layers, one global maximum pooling layer, and one fully connected layer, and the branch network model is configured to determine the parameter matrix of the corresponding fully connected layer.

In an embodiment, the feature extraction network model includes a three-dimensional residual neural network model.

In an embodiment, the image data quality evaluation method further includes:

acquiring a training data set, where the training data set includes a plurality of three-dimensional training image data; and

pretraining the image data quality evaluation network model according to the training data set to obtain the pretrained image data quality evaluation network model.

According to a second aspect, an image data quality evaluation apparatus is provided, including:

a first acquisition module, configured to acquire three-dimensional image data to be evaluated; and

a processing module, configured to input the three-dimensional image data to be evaluated into a pretrained image data quality evaluation network model for processing to obtain a quality evaluation score of the three-dimensional image data to be evaluated, where the pretrained image data quality evaluation network model includes a feature extraction network model, a hypernetwork model, and a regression model, the feature extraction network model is connected to the hypemetwork model and the regression model, and the hypernetwork model is connected to the regression model, where,

the feature extraction network model is configured to: process the three-dimensional image data to be evaluated to obtain a feature matrix, and send the feature matrix to the hypernetwork model and the regression model; the regression model includes a global maximum pooling layer and a plurality of fully connected layers; the hypemetwork model includes a plurality of branch network models that correspond one to one to the plurality of fully connected layers in the regression model; the hypemetwork model is configured to: process the feature matrix by using the plurality of branch network models to obtain parameter matrixes outputted by the plurality of branch network models, and correspondingly adjust parameters of the plurality of fully connected layers in the regression model according to the parameter matrixes to obtain an adjusted regression model; and the adjusted regression model is configured to process the feature matrix to obtain the quality evaluation score of the three-dimensional image data to be evaluated.

In an embodiment, the branch network model is formed by successively connecting two convolutional layers, one global maximum pooling layer, and one fully connected layer, and the branch network model is configured to determine the parameter matrix of the corresponding fully connected layer.

In an embodiment, the feature extraction network model includes a three-dimensional residual neural network model.

In an embodiment, the image data quality evaluation apparatus further includes: a second acquisition module, configured to acquire a training data set, where the training data set includes a plurality of three-dimensional training image data;

a pretrained module, configured to pretrain the image data quality evaluation network model according to the training data set to obtain the pretrained image data quality evaluation network model.

According to a third aspect, a terminal device is provided. The terminal device includes a memory, a processor, and a computer program stored in the memory and configured to be executed by the processor, where the processor is configured to execute the computer program to implement the foregoing image data quality evaluation method according to any implementation of the first aspect,

According to a fourth aspect, a computer-readable storage medium is provided. The computer-readable storage medium stores a computer program. The computer program, when executed by a processor, implements the foregoing image data quality evaluation method according to any implementation of the first aspect.

According to a fifth aspect, a computer program product is provided. When the computer program product runs on a terminal device, the terminal device is enabled to perform the foregoing image data quality evaluation method according to any implementation of the first aspect.

Beneficial Effects of the Present Invention

Beneficial Effects

Beneficial effects of the image data quality evaluation method provided in the embodiments of this application lie in that in the embodiments of this application, a three-dimensional image data to be evaluated is inputted into a pretrained image data quality evaluation network model including a feature extraction network model, a hypernetwork model, and a regression model. The three-dimensional image data to be evaluated is processed by using the feature extraction network model to obtain a feature matrix. The feature matrix is processed by using a plurality of branch network models that correspond one to one to a plurality of fully connected layers in the regression model in the hypemetwork model to obtain a plurality of parameter matrixes outputted by the plurality of branch network models. Correspondingly; parameters of the plurality of fully connected layers in the regression model are adjusted according to the plurality of parameter matrixes to obtain an adjusted regression model. The feature matrix is processed by using the adjusted regression model to obtain a quality evaluation score of the three-dimensional image data to be evaluated, to implement the dynamic adjustment of parameters of a model according to content of the inputted three-dimensional image data to be evaluated, to improve the processing efficiency and quality evaluation precision of image data.

### BRIEF DESCRIPTION OF THE DRAWINGS

### Brief Description of Drawings

To describe the technical solutions in the embodiments of this application more clearly, the following briefly introduces the accompanying drawings required for describing the embodiments or exemplary technologies. Apparently, the accompanying drawings in the following description show merely some embodiments of this application, and a person of ordinary skill in the art may still derive other drawings from these accompanying drawings without creative efforts.

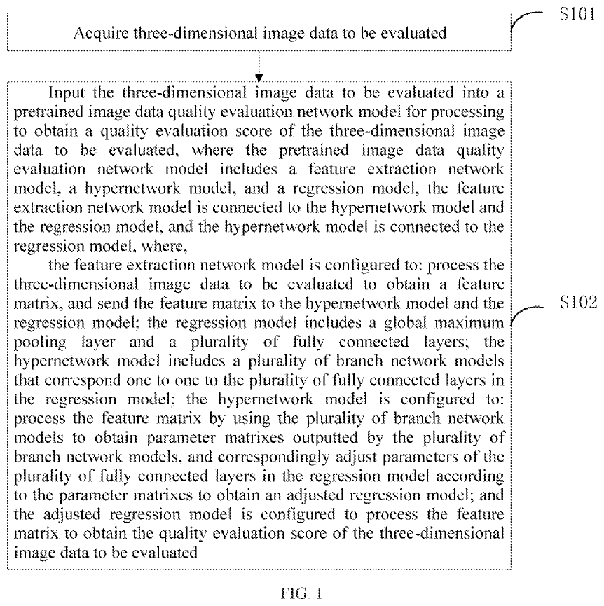

FIG. 1 is a schematic flowchart of an image data quality evaluation method according to an embodiment of this application;

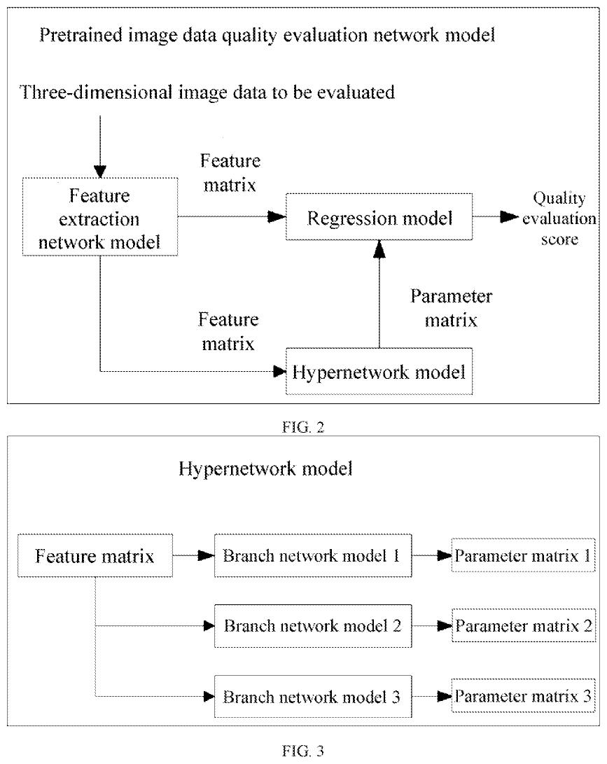

FIG. 2 is a schematic structural diagram of a pretrained image data quality evaluation network model according to an embodiment of this application;

FIG. 3 is a schematic structural diagram of a hypemetwork model according, to an embodiment of this application;

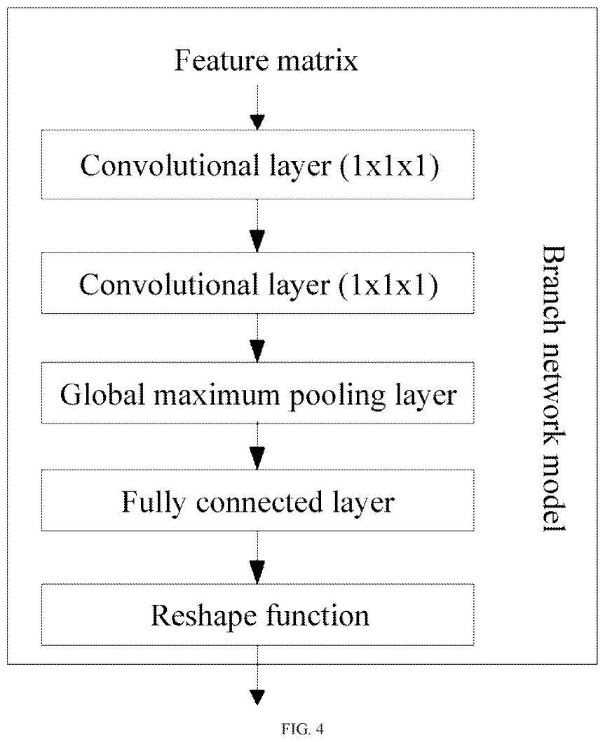

FIG. 4 is a schematic structural diagram of a branch network model according to an embodiment of this application;

FIG. 5 is a schematic structural diagram of an image data quality evaluation apparatus according to an embodiment of this application; and

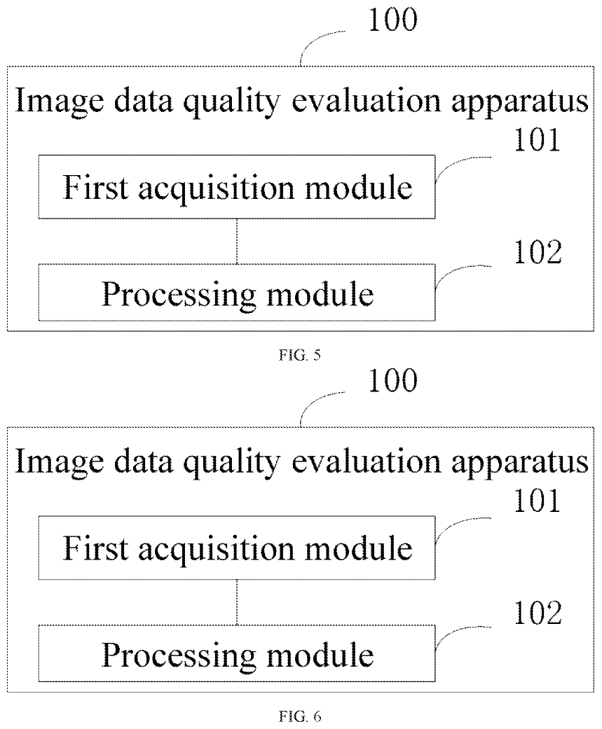

FIG. 6 is a schematic structural diagram of a terminal device according to an embodiment of this application.

### EMBODIMENTS OF THE PRESENT INVENTION

Implementations of the Present Invention

To make the objectives, technical solutions, and advantages of this application more comprehensible, this application is further described below in detail with reference to the accompanying drawings and embodiments. It should be understood that the specific embodiments described herein are merely used to describe this application rather than limiting this application.

It needs to be noted that when a component is “fastened to” or “disposed in” another component, it may be directly on the another component or indirectly on the another component. When a component is “connected” to another component, it may be connected directly or indirectly to the another component. The orientations or positional relationships indicated by the terms “up”, “down”, “left”, “right”, and the like are orientations or positional relationships based on the accompanying drawings, are merely for the ease of description, are not intended to indicate or imply that the apparatus or component must have a particular orientation or must be constructed and operated in a particular orientation, and therefore are not to be construed as a limitation of this application. A person of ordinary skill in the art may understand the specific meanings of the foregoing terms in accordance with the specific circumstances. The terms “first” and “second” are used only for ease of description, but are not intended to indicate or imply relative importance or implicitly specify a quantity of indicated technical features. The term “plurality of” means two or more, unless specifically and specifically limited otherwise.

To describe the technical solutions provided in this application, the following detailed description is provided in conjunction with specific accompanying drawings and embodiments.

The image data quality evaluation method provided in some embodiments of this application may be applied to terminal devices such as a cell phone, a tablet computer, a laptop computer, and an ultra-mobile personal computer (UMPC), and specific types of terminal devices are not limited in the embodiments of this application.

FIG. 1 is a schematic flowchart of an image data quality evaluation method according to this application. As an example rather imitation, the method may be plied to the foregoing laptop computer.

S101: Acquire three-dimensional image data to be evaluated.

During specific application, three-dimensional image data to be evaluated captured by a camera or transmitted by another terminal device is acquired. The three-dimensional image data to be evaluated includes three-dimensional image data or three-dimensional video data that requires quality evaluation. For example, in a clinical diagnosis process in the medical field, the three-dimensional image data to be evaluated is three-dimensional imaging data transmitted by a medical imaging device.

S102: Input the three-dimensional image data to be evaluated into a pretrained image data quality evaluation network model for processing to obtain a quality evaluation score of the three-dimensional image data to be evaluated, where the pretrained image data quality evaluation network model includes a feature extraction network model, a hypemetwork model, and a regression model, the feature extraction network model is connected to the hypernetwork model and the regression model, and the hypernetwork model is connected to the regression model, where,

the feature extraction network model is configured to: process the three-dimensional image data to be evaluated to obtain a feature matrix, and send the feature matrix to the hypernetwork model and the regression model; the regression model includes a global maximum pooling layer and a plurality of fully connected layers; the hypemetwork model includes a plurality of branch network models that correspond one to one to the plurality of fully connected layers in the regression model; the hypemetwork model is configured to: process the feature matrix by using the plurality of branch network models to obtain parameter matrixes outputted by the plurality of branch network models, and correspondingly adjust parameters of the plurality of fully connected layers in the regression model according to the parameter matrixes to obtain an adjusted regression model; and the adjusted regression model is configured to process the feature matrix to obtain the quality evaluation score of the three-dimensional image data to be evaluated.

During specific application, the three-dimensional image data to be evaluated is inputted into the pretrained image data quality evaluation network model for processing. The pretrained image data quality evaluation network model includes, but is not limited to, a feature extraction network model, a hypemetwork model, and a regression model. Feature extraction is performed on the inputted three-dimensional image data to be evaluated by using the feature extraction network model, to obtain a feature matrix outputted by the feature extraction network model, and the feature matrix is sent to the hypernetwork model and the regression model.

During specific application, the regression model includes, but is not limited to, one global maximum pooling layer and a plurality of fully connected layers. A quantity of the fully connected layers may be specifically set according to an actual requirement. For example, it is set that the regression model includes one global maximum pooling layer and three fully connected layers.

During specific application, the global maximum pooling layer in the regression model is configured to perform dimensionality reduction on an inputted high-dimensionality feature matrix, to obtain a processed two-dimensional feature matrix. The plurality of fully connected layers are configured to perform regression processing on the processed two-dimensional feature matrix, to obtain a quality evaluation score of the three-dimensional image data to be evaluated.

Specifically, the regression model is formed by cascading one global maximum pooling layer and a plurality of fully connected layers.

During specific application, the hypernetwork model includes a plurality of branch network models that correspond one to one to the plurality of fully connected layers in the regression model. Each branch network is configured to determine a parameter of a corresponding fully connected layer.

Specifically, the plurality of branch network models in the hypemetwork model process the feature matrix outputted by the feature extraction network model to obtain a plurality of parameter matrixes outputted by the plurality of branch network models. A parameter of a corresponding fully connected layer in the regression model is adjusted according to the parameter matrix outputted by the branch network model, to obtain an adjusted regression model. Regression processing is performed on the feature matrix outputted by the feature extraction network model by using the adjusted regression model to obtain the quality evaluation score of three-dimensional image data to be evaluated.

As an example rather than a limitation, a value range of the quality evaluation score is [4, 20]. The meaning of the value of the quality evaluation score may be specifically set according to a user requirement. For example, when the value of the quality evaluation score is set higher, the quality of the three-dimensional image data to be evaluated is higher.

It may be understood that after the feature extraction is performed on the three-dimensional image data to be evaluated, a dimensionality of the obtained feature matrix is higher than a dimensionality of the three-dimensional image data to be evaluated. For example, when the three-dimensional image data to be evaluated is inputted, the feature matrix outputted by the feature extraction network model is a five-dimensional feature matrix.

As shown in FIG. 2, a schematic structural diagram of a pretrained image data quality evaluation network model is provided.

In FIG. 2, the pretrained image data quality evaluation network model includes a feature extraction network model, a hypemetwork model, and a regression model. When three-dimensional image data to be evaluated is inputted into the pretrained image data quality evaluation network model, feature extraction is performed on the three-dimensional image data to be evaluated by using the feature extraction network model to obtain a corresponding feature matrix. The feature matrix outputted by the feature extraction network model is processed by using a plurality of branch network models in the hypemetwork model to obtain a plurality of parameter matrixes that correspond one to one to a plurality of fully connected layers in the regression model, and parameters of the plurality of fully connected layers in the regression model are adjusted by using the parameter matrixes to obtain an adjusted regression model. Regression processing is performed on the feature matrix outputted by the feature extraction network model by using the adjusted regression model to obtain a quality evaluation score of three-dimensional image data to be evaluated.

As shown in FIG. 3, a schematic structural diagram of a hypemetwork model is provided.

In FIG. 3, it is preset that a regression model includes three fully connected layers. Correspondingly, the hypemetwork model includes three branch network models. A feature matrix extracted by using a feature extraction network model is inputted into the three branch network models. The feature matrix is processed on the basis of the three branch network models to correspondingly obtain three parameter matrixes. Parameters of the three corresponding fully connected layers in the regression model are adjusted respectively according to the three parameter matrixes to obtain an adjusted regression model.

In an embodiment, the branch network model is formed by successively connecting two convolutional layers, one global maximum pooling layer, and one fully connected layer, and the branch network model is configured to determine the parameter matrix of the corresponding fully connected layer.

During specific application, the branch network model in the hypernetwork model is formed by successively connected two convolutional layers, one global maximum pooling layer, and one fully connected layer.

It may be understood that, the branch network model is configured to determine a parameter matrix of a fully connected layer in a regression model corresponding to the branch network model. That is, although input data is same, the branch network models have different output data.

As shown in FIG. 4, a schematic structural diagram of a branch network model is provided.

In FIG. 4, a branch network model of a hypemetwork model is formed by successively connecting two 1×1×1× convolutional layers, one global maximum pooling layer, one fully connected layer, and a reshape function. The reshape function is a function that converts a specified matrix into a matrix with a specific dimensionality in MATLAB, and a quantity of elements in the matrix remains unchanged. The function may readjust a row quantity, a column quantity, and a dimensionality of a matrix.

In an embodiment, the feature extraction network model includes a three-dimensional residual neural network model.

During specific application, based on image data with different dimensionalities, corresponding network structures used for performing feature extraction are different. It is set that the feature extraction network model includes a three-dimensional residual neural network model (3D ResNet50), to better adapt to inputted three-dimensional image data to be evaluated, and facilitate feature extraction on three-dimensional image data to be evaluated with rich spatial structure information to obtain a corresponding feature matrix.

In an embodiment, the method further includes:

acquiring a training data set, where the training data set includes a plurality of three-dimensional training image data; and

pretraining the image data quality evaluation network model according to the training data set to obtain the pretrained image data quality evaluation network model.

During specific application, the acquiring the training data set including the plurality of three-dimensional training image data and pretraining the image data quality evaluation network model on the basis of the training data set includes: performing training by using an end-to-end rotation process, randomly dividing each training image data into a plurality of patches (for example, into nine patches), using the patches of each training image data as input data, and outputting a quality evaluation score of the training image data During a test, for each training image data, the patches are cropped successively, it is set that a step size is half a size of a patch, and a quality evaluation score of each patch is predicted. Finally, an average value of quality evaluation scores of all patches of a same training image data is calculated and used as a quality evaluation score of the training image data.

Specifically, in a pretrained process, epoch is optimized by using a mean absolute error (MAE) as a loss function and by using an Adam optimizer.

It may be understood that because it is set that the three-dimensional image data to be evaluated includes three-dimensional image data, correspondingly used training data set should include a plurality of three-dimensional training image data.

In this embodiment, a three-dimensional image data to be evaluated is inputted into a pretrained image data quality evaluation network model including a feature extraction network model, a hypemetwork model, and a regression model. The three-dimensional image data to be evaluated is processed by using the feature extraction network model to obtain a feature matrix. The feature matrix is processed by using a plurality of branch network models that correspond one to one to a plurality of fully connected layers in the regression model in the hypemetwork model to obtain a plurality of parameter matrixes outputted by the plurality of branch network models. Correspondingly, parameters of the plurality of fully connected layers in the regression model are adjusted according to the plurality of parameter matrixes to obtain an adjusted regression model. The feature matrix is processed by using the adjusted regression model to obtain a quality evaluation score of the three-dimensional image data to be evaluated, to implement the dynamic adjustment of parameters of a model according to content of the inputted three-dimensional image data to be evaluated, to improve the processing efficiency and quality evaluation precision of image data.

It should be understood that sequence numbers of the steps do not mean an execution sequence in the foregoing embodiments. The execution sequence of the processes should be determined on the basis of functions and internal logic of the processes, and should not constitute any limitation on the implementation processes of embodiments of this application.

Corresponding to the image data quality evaluation method in the foregoing embodiment. FIG. 5 is a structural block diagram of an image data quality evaluation apparatus according to an embodiment of this application. To facilitate description, only a part related to the embodiments of this application is shown.

The present invention further provides another preferred embodiment of an image data quality evaluation apparatus. In this embodiment, the image data quality evaluation apparatus includes a processor. The processor is configured to execute the following program modules stored in a memory: a first acquisition module, configured to acquire three-dimensional image data to be evaluated; and

a processing module, configured to input the three-dimensional image data to be evaluated into a pretrained image data quality evaluation network model for processing to obtain a quality evaluation score of the three-dimensional image data to be evaluated, where the pretrained image data quality evaluation network model includes a feature extraction network model, a hypemetwork model, and a regression model, the feature extraction network model is connected to the hypemetwork model and the regression model, and the hypemetwork model is connected to the regression model, where,

the feature extraction network model is configured to: process the three-dimensional image data to be evaluated to obtain a feature matrix, and send the feature matrix to the hypernetwork model and the regression model; the regression model includes a global maximum pooling layer and a plurality of fully connected layers; the hypernetwork model includes a plurality of branch network models that correspond one to one to the plurality of fully connected layers in the regression model; the hypernetwork model is configured to: process the feature matrix by using the plurality of branch network models to obtain parameter matrixes outputted by the plurality of branch network models; and correspondingly adjust parameters of the plurality of fully connected layers in the regression model according to the parameter matrixes to obtain an adjusted regression model; and the adjusted regression model is configured to process the feature matrix to obtain the quality evaluation score of the three-dimensional image data to be evaluated.

Referring to FIG. 5, an image data quality evaluation apparatus 100 includes:

a first acquisition module 101, configured to acquire three-dimensional image data to be evaluated; and

a processing module 102, configured to input the three-dimensional image data to be evaluated into a pretrained image data quality evaluation network model for processing to obtain a quality evaluation score of the three-dimensional image data to be evaluated, where the pretrained image data quality evaluation network model includes a feature extraction network model, a hypernetwork model, and a regression model, the feature extraction network model is connected to the hypemetwork model and the regression model, and the hypernetwork model is connected to the regression model, where,

the feature extraction network model is configured to: process the three-dimensional image data to be evaluated to obtain a feature matrix, and send the feature matrix to the hypernetwork model and the regression model; the regression model includes a global maximum pooling layer and a plurality of fully connected layers; the hypernetwork model includes a plurality of branch network models that correspond one to one to the plurality of fully connected layers in the regression model; the hypernetwork model is configured to: process the feature matrix by using the plurality of branch network models to obtain parameter matrixes outputted by the plurality of branch network models; and correspondingly adjust parameters of the plurality of fully connected layers in the regression model according to the parameter matrixes to obtain an adjusted regression model; and the adjusted regression model is configured to process the feature matrix to obtain the quality evaluation score of the three-dimensional image data to be evaluated.

In an embodiment, the branch network model is formed by successively connecting two convolutional layers, one global maximum pooling layer, and one fully connected layer, and the branch network model is configured to determine the parameter matrix of the corresponding fully connected layer.

In an embodiment, the feature extraction network model includes a three-dimensional residual neural network model.

In an embodiment, the image data quality evaluation apparatus 100 further includes:

a second acquisition module, configured to acquire a training data set, where the training data set includes a plurality of three-dimensional training image data;

a pretrained module, configured to pretrain the image data quality evaluation network model according to the training data set to obtain the pretrained image data quality evaluation network model.

In this embodiment, a three-dimensional image data to be evaluated is inputted into a pretrained image data quality evaluation network model including a feature extraction network model, a hypemetwork model, and a regression model. The three-dimensional image data to be evaluated is processed by using the feature extraction network model to obtain a feature matrix. The feature matrix is processed by using a plurality of branch network models that correspond one to one to a plurality of fully connected layers in the regression model in the hypemetwork model to obtain a plurality of parameter matrixes outputted by the plurality of branch network models. Correspondingly, parameters of the plurality of fully connected layers in the regression model are adjusted according to the plurality of parameter matrixes to obtain an adjusted regression model. The feature matrix is processed by using the adjusted regression model to obtain a quality evaluation score of the three-dimensional image data to be evaluated, to implement the dynamic adjustment of parameters of a model according to content of the inputted three-dimensional image data to be evaluated, to improve the processing efficiency and quality evaluation precision of image data.

It should be noted that content such as information exchange between the foregoing apparatuses/units and an execution process thereof is based on a same concept as the method embodiments of this application. For details about specific functions and technical effects of the content, refer to the method embodiments. The details are not described herein again.

FIG. 6 is a schematic structural diagram of a terminal device according to this embodiment. As shown in FIG. 6, a terminal device 6 in the embodiment includes: at least one processor 60 (only one is shown in FIG. 6), a memory 61, and a computer program 62 which is stored in the memory 61 and can be run on the at least one processor 60. When executing the computer program 62, the processor 60 implements the steps in any foregoing embodiment of the image data quality evaluation method.

The terminal device 6 may be a computing device, for example, a desktop computer, a laptop computer, a palmtop computer, or a cloud server. The terminal device may include, but not only limited to, the processor 60 and the memory 61. A person skilled in the art may understand that FIG. 6 is merely an example of the terminal device 6, and does not constitute a limitation of the terminal device 6, which may include more or fewer components than shown in the figure, or a combination of some components, or different components, and may also include, for example, an input/output device, and a network access device.

For example, the processor 60 may be a central processing unit (CPU). The processor 60 may be another general-purpose processor, a digital signal processor (DSP), an application-specific integrated circuit (ASIC), a field-programmable gate array (FPGA) or another programmable logic device, a discrete gate or transistor logic device, a discrete hardware component, or the like. The general-purpose processor may be a microprocessor, or the processor may be any conventional processor or the like.

In some embodiments, the memory 61 may be an internal storage unit of the terminal device 6, for example, a hard disk or internal memory of the terminal device 6. In some other embodiments, the memory 61 may be an external storage device of the terminal device 6, for example, a plug-in hard drive, a smart media card (SMC), a secure digital (SD) card, a flash card, or the like provided on the terminal device 6. The memory 61 may include both the internal storage unit and the external storage device of the terminal device 6. The memory 61 is configured to store an operating system, an application program, a boot loader, data, and other programs, for example, program code for the computer program. The memory 61 may be used to temporarily store data that has been outputted or is to be outputted.

A person skilled in the art may clearly understand that for the purpose of convenient and brief descriptions, division into the foregoing functional units or modules is merely used as an example for descriptions. During actual application, the foregoing functions can be allocated to different functional units or modules for implementation according to a requirement, in other words, an inner structure of an apparatus is divided into different functional units or modules to implement all or a part of the functions described above. The functional units or modules in the embodiments may be integrated into one processing unit, or each of the units may exist alone physically, or two or more units are integrated into one unit. The integrated unit may be implemented in the form of hardware or a software functional unit. In addition, the specific names of the functional units and modules are only for the purpose of facilitating mutual differentiation, and are not used to limit the scope of protection of this application. For specific operating processes of the units or modules in the foregoing system, refer to the corresponding process in the foregoing method embodiments, and details are not described herein again.

An embodiment of this application further provides a computer-readable storage medium. The computer-readable storage medium stores a computer program. The computer program, when executed by a processor, may implement the steps in the foregoing method embodiments.

An embodiment of this application provides a computer program product. When the computer program product runs on a mobile terminal, the mobile terminal is enabled to implement the steps in the foregoing method embodiments when executing the computer program product.

The integrated units may be stored in a computer-readable storage medium when implemented as a software functional unit and sold or used as a stand-alone product. Based on this understanding, this application implements all or part of the procedures in the methods of the above embodiments, which may be accomplished by a computer program that instructs related hardware, and the computer program may be stored in a computer-readable storage medium. The computer program, when executed by a processor, may implement the steps of each of the above embodiments of the methods. The computer program includes computer program code. The computer program code may be in the form of source code, in the form of object code, in the form of an executable file or in some intermediate form, and the like. The computer-readable medium may at least include: any entity or device capable of carrying computer program code to a photographing apparatus/terminal device, a recording medium, a computer memory, a read-only memory (ROM), a random access memory (RAM), an electric carrier signal, a telecommunication signal, and a software distribution medium. Examples include a USB flash drive, a removable hard drive, a disk or a CD-ROM. In some jurisdictions, under legislation and patent practice, the computer-readable medium may not be an electric carrier signal or a telecommunication signal.

In the foregoing embodiments, the description of each embodiment has respective focuses. For a part that is not described in detail or recorded in an embodiment, refer to related descriptions in other embodiments.

A person of ordinary skill in the art may be aware that, in combination with the examples described in embodiments disclosed in this specification, units and algorithm steps may be implemented by electronic hardware or a combination of computer software and electronic hardware. Whether the functions are performed by hardware or software depends on particular applications and design constraints of the technical solutions. A person skilled in the art may use different methods to implement the described functions for each particular application, but it should not be considered that the implementation goes beyond the scope of this application.

In the embodiments provided in this application, it should be understood that the disclosed apparatus/network device and method may be implemented in other forms. For example, the described apparatus/network device embodiment is merely an example. For example, division into the modules or units is merely logical function division and may be other division during actual implementation. For example, a plurality of units or components may be combined or integrated into another system, or some features may be ignored or not performed. In addition, the displayed or discussed mutual couplings or direct couplings or communication connections may be implemented by using some interfaces. The indirect couplings or communication connections between the apparatuses or units may be implemented in electronic, mechanical, or another form,

The units described as separate parts may or may not be physically separate, and parts displayed as units may or may not be physical units, may be located in one position, or may be distributed on a plurality of network units. Some or all of the units may be selected based on actual requirements to achieve the objectives of the solutions of embodiments.

Described above are merely optional embodiments of this application, and are not intended to limit this application. person skilled in the art may make various changes and variations to this application. Within the spirit and principle of this application, any modifications, equivalent substitutions, improvements, and the like shall fall within the scope of claims of this application.

1. An image data quality evaluation method, comprising:
acquiring three-dimensional image data to be evaluated; and
inputting the three-dimensional image data to be evaluated into a pretrained image data quality evaluation network model for processing to obtain a quality evaluation score of the three-dimensional image data to be evaluated, wherein the pretrained image data quality evaluation network model comprises a feature extraction network model, a hypernetwork model, and a regression model, the feature extraction network model is connected to the hypernetwork model and the regression model, and the hypernetwork model is connected to the regression model, wherein,
the feature extraction network model is configured to: process the three-dimensional image data to be evaluated to obtain a feature matrix, and send the feature matrix to the hypernetwork model and the regression model; the regression model comprises a global maximum pooling layer and a plurality of fully connected layers; the hypernetwork model comprises a plurality of branch network models that correspond one to one to the plurality of fully connected layers in the regression model; the hypernetwork model is configured to: process the feature matrix by using the plurality of branch network models to obtain parameter matrixes outputted by the plurality of branch network models, and correspondingly adjust parameters of the plurality of fully connected layers in the regression model according to the parameter matrixes to obtain an adjusted regression model; and the adjusted regression model is configured to process the feature matrix to obtain the quality evaluation score of the three-dimensional image data to be evaluated.

2. The image data quality evaluation method according to claim 1, wherein the branch network model is formed by successively connecting two convolutional layers, one global maximum pooling layer, and one fully connected layer, and the branch network model is configured to determine the parameter matrix of the corresponding fully connected layer.

3. The image data quality evaluation method according to claim 1, wherein the feature extraction network model comprises a three-dimensional residual neural network model.

4. The image data quality evaluation method according to claim 1, wherein the method further comprises:
acquiring a training data set, wherein the training data set comprises a plurality of three-dimensional training image data; and pretraining the image data quality evaluation network model according to the training data set to obtain the pretrained image data quality evaluation network model.

5-8. (canceled)

9. A terminal device, comprising a memory, a processor, and a computer program which is stored in the memory and can be run on the processor, wherein when executing the computer program, the processor implements a method according to claim 1.

10. A computer-readable storage medium, wherein the computer-readable storage medium stores a computer program, and when the computer program is executed by a processor, implements a method according to claim 1.

11. The image data quality evaluation method according to claim 2, wherein the method further comprises:
acquiring a training data set, wherein the training data set comprises a plurality of three-dimensional training image data; and pretraining the image data quality evaluation network model according to the training data set to obtain the pretrained image data quality evaluation network model.

12. The image data quality evaluation method according to claim 3, wherein the method further comprises:
acquiring a training data set, wherein the training data set comprises a plurality of three-dimensional training image data; and pretraining the image data quality evaluation network model according to the training data set to obtain the pretrained image data quality evaluation network model.
